(19) United States
(12) Patent Application Publication
(10) Pub. No.: US 2005/0222186 A1
(43) Pub. Date: Oct. 6, 2005

(54) Substituted diaminopyrimidines

(75) Inventors: Monika Baudler, Konstanz (DE); Shripad S. Bhagwat, San Diego, CA (US); Paul E. Erdman, San Diego, CA (US); Florian Gantner, Osaka (JP); Moorthy S. Palanki, Encinitas, CA (US); Christian Schudt, Konstanz (DE); Josef Stadlwieser, Konstanz (DE); James Zapf, San Diego, CA (US)

Correspondence Address: NATH & ASSOCIATES PLLC, 1030 FIFTEENTH STREET, N.W., SIXTH FLOOR, WASHINGTON, DC, 20005, US

(73) Assignee: Altana Pharma AG, Konstanz (DE)

(21) Appl. No.: 10/517,594

(22) PCT Filed: Jun. 7, 2003
(86) PCT No.: PCT/EP03/06016

§ 371 Date: Dec. 13, 2004

Related U.S. Application Data
(60) Provisional application No. 60/388,312, filed on Jun. 14, 2002.

(30) Foreign Application Priority Data
Jun. 20, 2002 (EP) 02013675.0

(51) Int. Cl. A61K 31/506; C07D 43/14; C07D 413/14; C07D 417/14

(52) U.S. Cl. 514/275; 544/296; 544/324

(57) ABSTRACT

The invention relates to substituted diaminopyrimidine compounds, which are effective therapeutic compounds for treating diseases and disorders associated with those commonly treated by Protein Kinase C theta (PKCθ) inhibitors.

### BACKGROUND OF THE INVENTION

1. Field of the Invention

The inventive subject matter relates to substituted diaminopyrimidine compounds, which are effective therapeutic compounds for treating diseases and disorders associated with those commonly treated by Protein Kinase C theta (PKCθ) inhibitors.

2. Description of Related Art

In International Patent Application WO 99/65881, 6-membered heterocyclic compounds are disclosed which are said to be useful as hypoglycemic agents. International Patent Application WO 00/39108 discloses aromatic heterocyclic compounds, which are said to be useful as thrombin or factor Xa inhibitors. In International Patent Application WO 00/39101, pyrimidine compounds are disclosed which are said to be useful as anti-cancer agents. International Patent Application WO 01/00214 discloses pyrimidines, which are said to be useful as SRC kinase inhibitor compounds. In U.S. Pat. No. 6,159,982 2,4-diaminopyrimidine derivates are described as dopamine D4 receptor antagonists.

### BRIEF SUMMARY OF THE INVENTION

It has now been found that the compounds of the formula 1, which are described in more detail below, possess surprising and particularly advantageous properties.

One embodiment of the inventive subject matter relates to compounds of the formula 1, 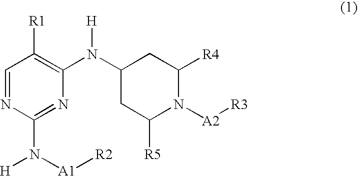
in which
R1 is a mono- or bicyclic aromatic radical substituted by R11, R12, R13 and R14, wherein R1 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl, where
   R11 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R12 methylenedioxy or ethylenedioxy,
   R12 is hydrogen, 1-4C-alkyl, 1-4C-alkoxy, 1-4C-alkoxycarbonyl, halogen, trifluoromethyl or hydroxyl, or together with R11 methylenedioxy or ethylenedioxy,
   R13 is hydrogen, 1-4C-alkyl or halogen and
   R14 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R2 is a mono- or bicyclic aromatic radical substituted by R21, R22, R23 and R24, wherein R2 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl,
   where
   R21 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R22 methylenedioxy or ethylenedioxy,
   R22 is hydrogen, 1-4C-alkyl, 1-4C-alkoxy, 1-4C-alkoxycarbonyl, halogen, trifluoromethyl or hydroxyl, or together with R21 methylenedioxy or ethylenedioxy,
   R23 is hydrogen, 1-4C-alkyl or halogen and
   R24 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R3 is a mono- or bicyclic aromatic radical substituted by R31, R32, R33 and R34, wherein R3 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl,
   where
   R31 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R32 methylenedioxy or ethylenedioxy,
   R32 is hydrogen, 1-4C-alkyl, 1-4C-alkoxy, 1-4C-alkoxycarbonyl, halogen, trifluoromethyl or hydroxyl, or together with R31 methylenedioxy or ethylenedioxy,
   R33 is hydrogen, 1-4C-alkyl or halogen and
   R34 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R4 is hydrogen or methyl,
R5 is hydrogen or methyl,
A1 is 1-3C-alkylene or ethyleneoxy (—CH₂—CH₂—O—) and
A2 is 1-3C-alkylene or ethyleneoxy (—CH₂—CH₂—O—),
   and their salts.

Another embodiment of the inventive subject matter relates to a pharmaceutical composition comprising a compound of the above formula and/or a pharmaceutically acceptable salt thereof together with pharmaceutically acceptable excipients and/or carrier.

A further embodiment of the inventive subject matter relates to a method of treating a patient afflicted with a disease or disorder, comprising the step of administering a therapeutically effective amount of a compound as described above and/or a pharmaceutically acceptable salt thereof to said patient afflicted with said disease or disorder, wherein the disease is selected from the group of acute and/or chronic airway disorders, inflammatory or allergen-induced airway disorder, bronchitis, obstructive bronchitis, spastic bronchitis, allergic bronchitis, allergic asthma, bronchial asthma, emphysema, chronic obstructive pulmonary disease (COPD), a disorder which is based on an excessive release of T-Cell derived cytokines, HIV-infection, septic shock, adult respiratory distress syndrome, graft-versus-host reactions, acute or chronic rejection of organ or tissue allo- or xenografts, generalized inflammations in the gastrointestinal area, rheumatoid arthritis, rheumatoid spondylitis, osteoarthritis, allergic and/or chronic, faulty immunological reactions in the area of the upper airways and the adjacent regions, dermatose of the proliferative, inflammatory or allergic type, psoriasis (vulgaris), toxic and allergic contact eczema, atopic eczema, seborrheic eczema, lichen simplex, sunburn, pruritus in the anogenital area, alopecia areata, hypertrophic scars, discoid lupus erythematosus, follicular and vide-area pyodermias, endogenous and exogenous acne, acne rosacea, other proliferative, inflammatory, allergic skin disorders, a disorder in connection with disturbances of brain metabolism or alternatively disorders of the central nervous system (CNS), cerebral senility, senile dementia, multiinfarct dementia, depression, arteriosclerotic dementia, cancer and diabetes insipidus.

A still further embodiment of the inventive subject matter relates to a process for preparing a compound of formula 1 as described above or a salt thereof, which comprises reacting a boronic acid derivative R1-B(OH)₂ wherein R1 has the meaning specified above, with a pyrimidine derivate of formula (4) 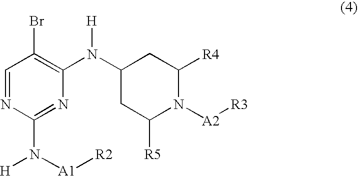
in which A1, A2, R2, R3, R4 and R5 have a meaning specified above, and optionally converting an obtained compound into a corresponding salt or converting an obtained salt into a corresponding free compound.

### DETAILED DESCRIPTION OF THE INVENTION

Definitions

The following terms are used herein to have the indicated meanings and are capable of including additional components well known to one of ordinary skill in the art.

1-4C-Alkyl represents straight-chain or branched alkyl radicals having 1 to 4 carbon atoms. Examples which may be mentioned are the butyl, isobutyl, sec-butyl, tert-butyl, propyl, isopropyl, ethyl and the methyl radical.

Hydroxy-1-4C-alkyl represents aforementioned 1-4C-alkyl radicals, which are substituted by a hydroxy group. Examples which may be mentioned are the hydroxymethyl, the 2-hydroxyethyl and the 3-hydroxypropyl radical.

1-4C-Alkoxy represents radicals, which in addition to the oxygen atom contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radical.

2-4C-Alkenyl represents straight-chain or branched alkenyl radicals having 2 to 4 carbon atoms. Examples which may be mentioned are the 2-butenyl, 3-butenyl, 1-propenyl and the 2-propenyl radical (allyl radical).

2-4C-Alkenyloxy represents a radical, which in addition to the oxygen atom contains a 2-4C-alkenyl radical. An example which may be mentioned is the allyloxy radical.

1-4C-Alkylcarbonyl represents a radical, which in addition to the carbonyl group contains one of the aforementioned 1-4C-alkyl radicals. An example which may be mentioned is the acetyl radical.

Carboxyl is the group —COOH.

Aminocarbonyl is Amino (—NH₂) which is bound to the carbonyl group, i.e. aminocarbonyl is —CO—NH₂. Mono- or di-1-4C-alkylamino radicals contain, in addition to the nitrogen atom, one or two of the aforementioned 1-4C-alkyl radicals. Di-1-4C-alkylamino is preferred and here, in particular, dimethyl-, diethyl or diisopropylamino.

Mono- or di-1-4C-alkylaminocarbonyl represents a radical, which in addition to the carbonyl group contains one of the aforementioned mono- or di-1-4C-alkylamino radicals.

1-4C-Alkoxycarbonyl represents a carbonyl group, to which one of the aforementioned 1-4C-alkoxy radicals is bonded. Examples which may be mentioned are the methoxycarbonyl (CH₃O—C(O)—) and the ethoxycarbonyl radical (CH₃CH₂O—C(O)—).

Carboxy-1-4C-alkyl for example represents the carboxymethyl (—CH₂COOH) or the carboxyethyl radical (—CH₂CH₂COOH).

1-4C-Alkoxycarbonyl-1-4C-alkyl represents one of the aforementioned 1-4C-alkyl radicals, which is substituted by one of the aforementioned 1-4C-alkoxycarbonyl radicals. An example which may be mentioned is the ethoxycarbonylmethyl radical (CH₃CH₂OC(O)CH₂—).

Halogen within the meaning of the invention is bromo, chloro and fluoro.

Aryl-1-4C-alkyl represents an aryl-substituted 1-4C-alkyl radical. An example which may be mentioned is the benzyl radical.

Aryl-1-4C-alkoxy represents an aryl-substituted 1-4C-alkoxy radical. An example which may be mentioned is the benzyloxy radical.

1-4C-Alkylcarbonylamino represents an amino group to which a 1-4C-alkylcarbonyl radical is bonded. Examples which may be mentioned are the propionylamino (C₃H₇C(O)NH—) and the acetylamino radical (acetamido radical) (CH₃C(O)NH—).

1-4C-Alkoxycarbonylamino represents an amino radical, which is substituted by one of the aforementioned 1-4C-alkoxycarbonyl radicals. Examples which may be mentioned are the ethoxycarbonylamino and the methoxycarbonylamino radical.

1-4C-Alkoxy-1-4C-alkoxy represents one of the aforementioned 1-4C-alkoxy radicals, which is substituted by a further 1-4C-alkoxy radical. Examples which may be mentioned are the radicals 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxycarbonyl represents a carbonyl group, to which one of the aforementioned 1-4C-alkoxy-1-4C-alkoxy radicals is bonded. Examples which may be mentioned are the 2-(methoxy)-ethoxycarbonyl (CH₃—O—CH₂CH₂—O—CO—) and the 2-(ethoxy)ethoxycarbonyl radical (CH₃CH₂—O—CH₂CH₂—O—CO—).

1-4C-Alkoxy-1-4C-alkoxycarbonylamino represents an amino radical, which is substituted by one of the aforementioned 1-4C-alkoxy-1-4C-alkoxycarbonyl radicals. Examples which may be mentioned are the 2-(methoxy)ethoxycarbonylamino and the 2-(ethoxy)ethoxycarbonylamino radical.

In case that R11 together with R12, or R21 together with R22, or R31 together with R32 form a methylenedioxy (—O—CH₂—O—) or ethylenedioxy (—O—CH₂CH₂—O—) group, it is necessary that R11 and R12, or R21 and R22, or R31 and R32 are in adjacent positions to each other (ortho-position).

1-3C-Alkylene represents straight-chain or branched 1-3C-alkylene radicals, for example the methylene (—CH₂—), ethylene (—CH₂CH₂—), ethylidene [—CH(CH₃)—], trimethylene (—CH₂CH₂CH₂—), isopropylidene [—C(CH₃)₂—] and the 1-methylethylene [—CH(CH₃)—CH₂ ] radical.

The compounds according to the invention have valuable pharmacological properties, which make them commercially utilizable. In one possible mode of action they may act as selective Protein Kinase C theta (PKCθ) inhibitors. As such they are suitable as therapeutics especially for the treatment of disorders, in particular of inflammatory nature, e.g. of the airways (asthma prophylaxis), of the skin, of the central nervous system, of the intestine, of the eyes and of the joints, which are mediated by T-cells and derived mediators such as cytokines, interleukins, chemokines, alpha-, beta- and gamma-interferon or tumor necrosis factor (TNF). The compounds according to the invention are distinguished here by low toxicity, good enteral absorption (high bioavailability), a large therapeutic breadth and the absence of significant side-effects.

On account of their PKC-inhibiting properties, the compounds according to the invention can be employed in human and veterinary medicine and therapeutics, where they can be used, for example, for the treatment and prophylaxis of the following illnesses: acute and chronic (in particular inflammatory and allergen-induced) airway disorders of various origins (bronchitis, obstructive bronchitis, spastic bronchitis, allergic bronchitis, allergic asthma, bronchial asthma, emphysema, COPD); dermatoses (especially of proliferative, inflammatory and allergic type) such as, for example, psoriasis (vulgaris), toxic and allergic contact eczema, atopic eczema, seborrheic eczema, lichen simplex, sunburn, pruritus in the anogenital area, alopecia areata, hypertrophic scars, discoid lupus erythematosus, follicular and wide-area pyodermias, endogenous and exogenous acne, acne rosacea and other proliferative, inflammatory and allergic skin disorders; disorders which are based on an excessive release of T-cell derived cytokines, e.g. disorders of the arthritis type (rheumatoid arthritis, rheumatoid spondylitis, osteoarthritis and other arthritic conditions), disorders of the immune system (AIDS, multiple sclerosis), HIV-infection, septic shock or adult respiratory distress syndrome, graft-versus-host reactions, acute or chronic rejection of organ or tissue allo- or xenografts, and generalized inflammations in the gastrointestinal area (Crohn's disease and ulcerative colitis); disorders which are based on allergic and/or chronic, faulty immunological reactions in the area of the upper airways (pharynx, nose) and the adjacent regions (paranasal sinuses, eyes), such as, for example, allergic rhinitis/sinusitis, chronic rhinitis/sinusitis, allergic conjunctivitis and nasal polyps autoimmune disorders involving various tissues (e.g. kidney, pancreas, thyroidea, skin or joints). In addition, the compounds according to the invention can be employed for the treatment of cancer, diabetes insipidus and disorders in connection with disturbances of brain metabolism, such as, for example, cerebral senility, senile dementia (Alzheimer's dementia), multiinfarct dementia or alternatively disorders of the CNS, such as, for example, depressions or arteriosclerotic dementia.

The compounds according to the invention may be administered as the sole active ingredient or together, i.e. in a fixed or free combination, with other therapeutic agents used in clinical practice for the treatment of those diseases listed above. Reference is made in this connection to other drugs in immunomodulating regimens or other anti-inflammatory agents e.g. for the treatment or prevention of inflammatory or autoimmune disorders or allo- or xenograft acute or chronic rejection. For example, the compounds of formula 1 may be used in combination with cyclosporines or ascomycines or their immunosuppressive analogs or derivatives; an mTOR inhibitor, corticosteroids; cyclophosphamide; azathioprene; methotrexate; an accelerating lymphocyte homing agent; leflunomide or analogs thereof, mizoribine; mycophenolic acid; mycophenolate mofetil; 15-deoxyspergualine or analogs thereof; immunosuppressive monoclonal antibodies, e.g. monoclonal antibodies to leukocyte receptors or their ligands; or other immunomodulatory compounds, e.g. a recombinant binding molecule or portions of it e.g. CTLA4 or other adhesion molecule inhibitors, e.g. mAbs or low molecular weight inhibitors including LFA-1 antagonists, Selectin antagonists and VLA-4 antagonists. Compounds according to this invention may also be administered together with an anti-proliferative drug, e.g. a chemotherapeutic drug, e.g. in cancer treatment, or with an anti-diabetic drug in diabetes treatment.

The invention further relates to the compounds according to the invention for use in the treatment of mammals, including man, which are suffering from one of the abovementioned illnesses. The process comprises administering to the sick mammal a therapeutically efficacious and pharmacologically tolerable amount of one or more of the compounds and/or a pharmaceutically acceptable salt thereof according to the invention.

The invention further relates to the compounds according to the invention for use in the treatment and/or prophylaxis of illnesses, in particular the illnesses mentioned.

The invention likewise relates to the use of the compounds according to the invention for the production of pharmaceutical compositions which are employed for the treatment and/or prophylaxis of the illnesses mentioned.

Pharmaceutical compositions for the treatment and/or prophylaxis of the illnesses mentioned, which contain one or more of the compounds according to the invention, are furthermore a subject of the invention.

The inventive subject matter relates to compounds of the formula 1, 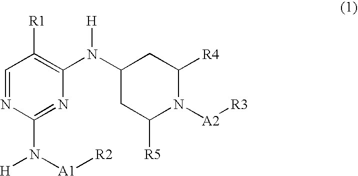
in which
R1 is a mono- or bicyclic aromatic radical substituted by R11, R12, R13 and R14, wherein R1 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl, where
   R11 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R12 methylenedioxy or ethylenedioxy,
   R12 is hydrogen, 1-4C-alkyl, 1-4C-alkoxy, 1-4C-alkoxycarbonyl, halogen, trifluoromethyl or hydroxyl, or together with R11 methylenedioxy or ethylenedioxy,
   R13 is hydrogen, 1-4C-alkyl or halogen and
   R14 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R2 is a mono- or bicyclic aromatic radical substituted by R21, R22, R23 and R24, wherein R2 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl,
   where
   R21 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R22 methylenedioxy or ethylenedioxy,
   R22 is hydrogen, 1-4C-alkyl, 1-4C-alkoxy, 1-4C-alkoxycarbonyl, halogen, trifluoromethyl or hydroxyl, or together with R21 methylenedioxy or ethylenedioxy,
   R23 is hydrogen, 1-4C-alkyl or halogen and
   R24 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R3 is a mono- or bicyclic aromatic radical substituted by R31, R32, R33 and R34, wherein R3 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl,
   where
   R31 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R32 methylenedioxy or ethylenedioxy,
   R32 is hydrogen, 1-4C-alkyl, 1-4C-alkoxy, 1-4C-alkoxycarbonyl, halogen, trifluoromethyl or hydroxyl, or together with R31 methylenedioxy or ethylenedioxy,
   R33 is hydrogen, 1-4C-alkyl or halogen and
   R34 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R4 is hydrogen or methyl,
R5 is hydrogen or methyl,
A1 is 1-3C-alkylene or ethyleneoxy (—CH₂—CH₂—O—) and
A2 is 1-3C-alkylene or ethyleneoxy (—CH₂—CH₂—O—),
   and their salts.

Another embodiment of the Inventive subject matter relates to a compound of formula 1,

in which

R1 is a mono- or bicyclic aromatic radical substituted by R11, R12, R13 and R14, wherein R1 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl, where
   R11 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R12 is hydrogen, 1-4C-alkyl, 1-4C-alkoxy, 1-4C-alkoxycarbonyl, halogen, trifluoromethyl or hydroxyl,
   R13 is hydrogen, 1-4C-alkyl or halogen and
   R14 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R2 is a mono- or bicyclic aromatic radical substituted by R21, R22, R23 and R24, wherein R2 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl,
   where
   R21 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R22 is hydrogen, 1-4C-alkyl, 1-4C-alkoxy, 1-4C-alkoxycarbonyl, halogen, trifluoromethyl or hydroxyl,
   R23 is hydrogen, 1-4C-alkyl or halogen and
   R24 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R3 is a mono- or bicyclic aromatic radical substituted by R31, R32, R33 and R34, wherein R3 is selected from the group consisting of phenyl, naphthyl, pyrrolyl, pyrazolyl, imidazolyl, 1,2,3-triazolyl, indolyl, benzimidazolyl, furanyl (furyl), benzofuranyl(benzofuryl), thiophenyl(thienyl), benzothiophenyl (benzothienyl), thiazolyl, isoxazolyl, pyridinyl, pyrimidinyl, quinolinyl and isoquinolinyl,
   where
   R31 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R32 is hydrogen, 1-4C-alkyl, 1-4C-alkoxy, 1-4C-alkoxycarbonyl, halogen, trifluoromethyl or hydroxyl,
   R33 is hydrogen, 1-4C-alkyl or halogen and
   R34 is hydrogen, 1-4C-alkyl or halogen,
   where
   aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R4 is hydrogen,
R5 is hydrogen,
A1 denotes 1-3C-alkylene and
A2 denotes 1-3C-alkylene,
   and their salts.

An embodiment of the inventive subject matter, to be emphasized is a compound of formula 1,

in which

R1 is an aromatic radical substituted by R11, R12, R13 and R14, wherein R1 is selected from the group consisting of phenyl, furanyl (furyl) and thiophenyl(thienyl), where
   R11 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R12 methylenedioxy or ethylenedioxy,
   R12 is hydrogen or halogen, or together with R11 methylenedioxy or ethylenedioxy,
   R13 is hydrogen and
   R14 is hydrogen,
R2 is an aromatic radical substituted by R21, R22, R23 and R24, wherein R2 is selected from the group consisting of pyridinyl and pyrimidinyl,
   where
   R21 is hydrogen, 1-4C-alkyl, 1-4C-alkoxy or halogen,
   R22 is hydrogen or halogen,
   R23 is hydrogen and
   R24 is hydrogen,
R3 is an aromatic radical substituted by R31, R32, R33 and R34, wherein R3 is selected from the group consisting of phenyl and pyridinyl,
   where
   R31 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R32 is hydrogen or halogen,
   R33 is hydrogen and
   R34 is hydrogen,
R4 is hydrogen or methyl,
R5 is hydrogen or methyl,
A1 is 1-3C-alkylene or ethyleneoxy (—CH₂—CH₂—O—) and
A2 is 1-3C-alkylene or ethyleneoxy (—CH₂—CH₂—O—),
   and their salts.

An embodiment of the inventive subject matter to be particularly emphasized, is a compound of formula 1,

in which

R1 is an aromatic radical substituted by R11, R12, R13 and R14, wherein R1 is selected from the group consisting of phenyl, furanyl (furyl) and thiophenyl(thienyl),
   where
   R11 is hydrogen, 1-4C-alkoxy, 1-4C-alkylcarbonyl, carboxyl, aminocarbonyl, mono- or di-1-4C-alkylaminocarbonyl, halogen, hydroxyl or mono- or di-1-4C-alkylamino, 1-4C-alkylcarbonylamino, or together with R12 methylenedioxy or ethylenedioxy,
   R12 is hydrogen or halogen, or together with R11 methylenedioxy or ethylenedioxy,
   R13 is hydrogen and
   R14 is hydrogen,
R2 is an aromatic radical substituted by R21, R22, R23 and R24, wherein R2 is selected from the group consisting of pyridinyl and pyrimidinyl,
   where
   R21 is hydrogen,
   R22 is hydrogen,
   R23 is hydrogen and
   R24 is hydrogen,
R3 is an aromatic radical substituted by R31, R32, R33 and R34, wherein R3 is selected from the group consisting of phenyl and pyridinyl,
   where
   R31 is hydrogen, 1-4C-alkoxy or halogen,
   R32 is hydrogen,
   R33 is hydrogen and
   R34 is hydrogen,
R4 is hydrogen or methyl,
R5 is hydrogen or methyl,
A1 denotes methylene, ethylene, ethylidene [—CH(CH₃)—] or ethyleneoxy (—CH₂—CH₂—O—) and
A2 denotes methylene, ethylene, ethylidene [—CH(CH₃)—] or ethyleneoxy (—CH₂—CH₂—O—),
   and their salts.

Selected compounds of formula 1 are those,

in which

R1 is furanyl (furyl), thiophenyl(thienyl) or phenyl substituted by R11 and R12,
   where
   R11 is hydrogen, 1-4C-alkoxy, carboxyl, aminocarbonyl, halogen or di-1-4C-alkylamino and
   R12 is hydrogen,
R2 is pyridinyl,
R3 is phenyl,
R4 is hydrogen,
R5 is hydrogen,
A1 denotes methylene and
A2 denotes methylene,
   and their salts.

Further selected compounds of formula 1 are those,

in which

R1 is furanyl (furyl), thiophenyl(thienyl) or phenyl substituted by R11 and R12,
   where
   R11 is hydrogen, 1-4C-alkoxy, 1-4C-alkylcarbonyl, carboxyl, aminocarbonyl, mono- or di-1-4C-alkylaminocarbonyl, halogen, hydroxyl or mono- or di-1-4C-alkylamino, 1-4C-alkylcarbonylamino, or together with R12 methylenedioxy or ethylenedioxy,
   R12 is hydrogen or halogen, or together with R11 methylenedioxy or ethylenedioxy,
R2 is pyridinyl,
R3 is phenyl,
R4 is hydrogen,
R5 is hydrogen,
A1 denotes methylene and
A2 denotes methylene,
   and their salts.

Exemplary substituents R1 are: 2-furyl, 3-furyl, 2-thienyl, 3-thienyl, phenyl, 4-methoxyphenyl, 4-chlorophenyl, 4-dimethylaminophenyl, 4-aminocarbonylphenyl, 4-carboxyphenyl, 3-chloro-4-fluorophenyl, 3-acetylaminophenyl, benzo[1,3]dioxol-5-yl, 3-hydroxyphenyl, 4-hydroxyphenyl, 4-acetylphenyl, 3-acetyl-phenyl, 4-acetylaminophenyl, 4-dimethylaminocarbonyl-phenyl and 4-aminocarbonylphenyl.

In a preferred embodiment the inventive subject matter relates to a compound of formula 1, wherein R1 is furanyl (furyl), thiophenyl(thienyl) or phenyl substituted by R11 and R12, where R11 is hydrogen, 1-4C-alkoxy, carboxyl, aminocarbonyl, halogen or di-1-4C-alkylamino and R12 is hydrogen.

In another preferred embodiment the inventive subject matter relates to a compound of formula 1, wherein R1 is 2-furanyl or 3-furanyl.

In a still preferred embodiment the inventive subject matter relates to a compound of formula 1, wherein R1 is 2-thiophenyl or 3-thiophenyl.

In another still preferred embodiment the inventive subject matter relates to a compound of formula 1, wherein R1 is selected from the group of phenyl, 4-methoxyphenyl, 4-chlorophenyl, 4-dimethylaminophenyl, 4-aminocarbonylphenyl, 4-carboxyphenyl, 3-chloro-4-fluorophenyl, 3-acetylaminophenyl, benzo[1,3]dioxol-5-yl, 3-hydroxyphenyl, 4-hydroxyphenyl, 4-acetylphenyl, 3-acetylphenyl, 4-acetylaminophenyl, 4-dimethylaminocarbonyl-phenyl and 4-aminocarbonylphenyl.

Exemplary substituents R2 are: 4-pyridyl, 2-pyridyl, 3-pyridyl and 4-pyrimidinyl. Amongst the pyridyl groups, the 2-pyridyl group is preferred.

In another still preferred embodiment the inventive subject matter relates to a compound of formula 1, wherein R2 is selected from the group of 4-pyridyl, 2-pyridyl, 3-pyridyl and 4-pyrimidinyl.

Exemplary substituents A1-R2 are: 2-pyridylmethyl, 4-pyridylmethyl, 3-pyridylmethyl, 4-pyrimidinylmethyl, 2-pyridyl-1-ethyl, 2-pyridyl-2-ethyl, 3-pyridyl-2-ethyl, 4-pyrimidinyl-2-ethyl, 2-pyridyloxy-2-ethyl and 3-pyridyloxy-2-ethyl.

In another still preferred embodiment the inventive subject matter relates to a compound of formula 1, wherein A1-R2 is selected from the group of 2-pyridylmethyl, 4-pyridylmethyl, 3-pyridylmethyl, 4-pyrimidinylmethyl, 2-pyridyl-1-ethyl, 2-pyridyl-2-ethyl, 3-pyridyl-2-ethyl, 4-pyrimidinyl-2-ethyl, 2-pyridyloxy-2-ethyl and 3-pyridyloxy-2-ethyl.

Exemplary substituents R3 are: phenyl, 4-fluorophenyl, 4-methoxyphenyl, and 4-pyridinyl.

In another still preferred embodiment the inventive subject matter relates to a compound of formula 1, wherein R3 is selected from the group of phenyl, 4-fluorophenyl, 4-methoxyphenyl, and 4-pyridinyl.

Exemplary substituents A2-R3 are: benzyl, 4-fluorobenzyl, 4-methoxybenzyl, phenyl-1-ethyl, phenyl-2-ethyl and phenoxy-2-ethyl.

In another still preferred embodiment the inventive subject matter relates to a compound of formula 1, wherein A2-R3 is selected from the group of benzyl, 4-fluorobenzyl, 4-methoxybenzyl, phenyl-1-ethyl, phenyl-2-ethyl and phenoxy-2-ethyl.

The compounds according to the invention can be prepared as exemplary described in the paragraph “Examples” which follows below, or using analogous process steps starting from appropriate starting compounds. The compounds according to the invention can be prepared for example starting from appropriate 2,4,5-trihalopyrimidines, for example from 5-bromo-2,4-dichloropyrimidine, according to the following reaction scheme:

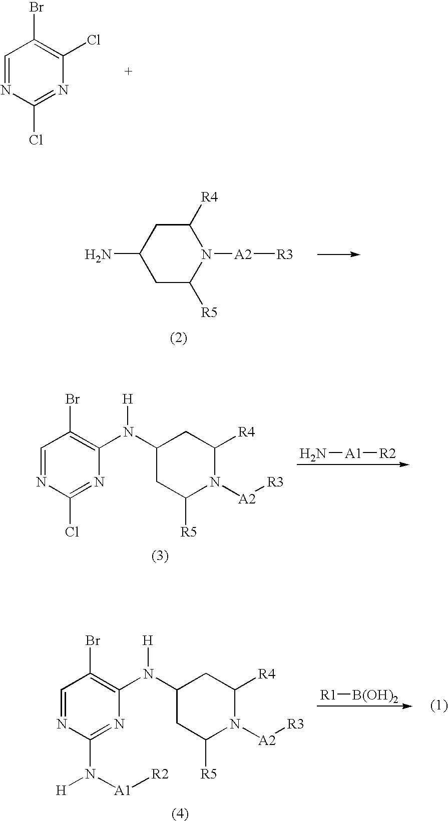

5-Bromo-2,4-dichloropyrimidine is reacted with the aminopiperidine 2 in a manner known per se. Advantageously, the reaction is carried out in an inert solvent at an appropriate temperature, such as room temperature, in the presence of a base (e.g. of an inorganic hydroxide, such as sodium hydroxide, or of an inorganic carbonate, such as potassium carbonate, or of an organic nitrogen base, such as triethylamine) or with an excess of compound 2. The subsequent reaction with the amine H₂N-A1-R2 is likewise carried out in the presence of an auxiliary base or with an excess of the amine, preferably at temperatures higher than room temperature, e.g. between 60 and 150° C., in particular at the boiling point of the inert solvent used. The concluding reaction with the boronic acid R1-B(OH)₂ is also carried out in a manner known per se to the person skilled in the art and familiar with the Suzuki reaction, e.g. as outlined in the Examples which follow below.

The starting compounds are known or can be prepared analogously to the known compounds. The substances according to the invention are isolated and purified in a manner known per se, for example, by distilling off the solvent in vacuo and recrystallizing the residue obtained from a suitable solvent or subjecting it to one of the customary purification methods, such as, for example, column chromatography on suitable support material.

Salts are obtained by dissolving the free compound in a suitable solvent, e.g. in a chlorinated hydrocarbon, such as dichloromethane or chloroform, or a low molecular weight aliphatic alcohol (ethanol, isopropanol) which contains the desired acid, or to which the desired acid is subsequently added. The salts are obtained by filtering, reprecipitating, precipitating with a non-solvent for the addition salt or by evaporating the solvent. Salts obtained can be converted by alkalization or by acidification into the free compounds, which in turn can be converted into salts. In this way, salts pharmaceutically not acceptable can be converted into pharmaceutically acceptable salts.

Possible salts of compounds of the formula 1—depending on substitution—are especially all acid addition salts. Particular mention may be made of the pharmaceutically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are used in salt preparation—depending on whether a mono- or polybasic acid is concerned and on which salt is desired—in an equimolar quantitative ratio or one differing therefrom.

Salts, which are pharmaceutically not acceptable, which can initially be obtained, for example, as process products in the production of the compounds according to the invention on the industrial scale, are converted into the pharmaceutically acceptable salts by processes known to the person skilled in the art.

It is known to the person skilled in the art that the compounds according to invention and their salts, if, for example, they are isolated in crystalline form, can contain various amounts of solvents. The invention therefore also comprises all solvates and in particular all hydrates of the compounds of the formula 1, and also all solvates and in particular all hydrates of the salts of the compounds of the formula 1.

In case of A1 and/or A2 being ethylidene [—CH(CH₃)—], the compounds of the formula 1 have one or two chiral centers. The invention relates to all four conceivable stereoisomers in any desired mixing ratio with one another, including the pure enantiomers, which are a preferred subject of the invention and which can be synthesized by using the corresponding optically pure starting compounds.

A further subject of the invention is a commercial product, consisting of a customary secondary pack, a primary pack containing the pharmaceutical composition (for example an ampoule or a blister pack) and, if desired, a pack insert, the medicament exhibiting antagonistic action against Protein Kinase C theta (PKCθ) and leading to the attenuation of the symptoms of illnesses which are connected Protein Kinase C theta (PKCθ), and the suitability of the medicament for the prophylaxis or treatment of illnesses which are connected with Protein Kinase C theta (PKCθ) being indicated on the secondary pack and/or on the pack insert of the commercial product, and the medicament containing one or more compounds of the formula I according to the invention. The secondary pack, the primary pack containing the medicament and the pack insert otherwise comply with what would be regarded as standard to the person skilled in the art for pharmaceutical compositions of this type.

The pharmaceutical compositions are prepared by processes, which are known per se and familiar to the person skilled in the art As pharmaceutical compositions, the compounds according to the invention (=active compounds) are either employed as such, or preferably in combination with suitable pharmaceutical excipients, e.g. in the form of tablets, coated tablets, capsules, suppositories, patches, emulsions, suspensions, gels or solutions, the active compound content advantageously being between 0.1 and 95%.

The person skilled in the art is familiar on the basis of his/her expert knowledge with the excipients, which are suitable for the desired pharmaceutical formulations. In addition to solvents, gel-forming agents, ointment bases and other active compound vehicles, it is possible to use, for example, antioxidants, dispersants, emulsifiers, preservatives, solubilizers or permeation promoters.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk@, Turbohaler® or the inhaler described in European Patent Application EP 0 505 321), using which an optimal administration of active compound can be achieved.

For the treatment of dermatoses, the compounds according to the invention are in particular used in the form of those pharmaceutical compositions, which are suitable for topical application. For the production of the pharmaceutical compositions, the compounds according to the invention (=active compounds) are preferably mixed with suitable pharmaceutical excipients and further processed to give suitable pharmaceutical formulations. Suitable pharmaceutical formulations, which may be mentioned are, for example, powders, emulsions, suspensions, sprays, oils, ointments, fatty ointments, creams, pastes, gels or solutions.

Pharmaceutical compositions according to the invention can be prepared by processes known per se. Dosage of the active compounds takes place in the order of magnitude customary for PKCθ inhibitors. Thus topical application forms (such as, for example, ointments) for the treatment of dermatoses contain the active compounds in a concentration of, for example, 0.1-99%. The dose for administration by inhalation is customarily between 0.1 and 3 mg per day. The customary dose in the case of systemic therapy (p.o. or i.v.) is between 0.03 and 3 mg per kilogram per day.

The following examples are illustrative of the present invention and are not intended to be limitations thereon. Likewise, further compounds of the formula 1 whose preparation is not described explicitly can be prepared analogously or in a manner familiar to the person skilled in the art using customary process techniques. The abbreviation ESMS stands for Electro Spray Mass Spectroscopy and eq stands for equivalent(s).

### EXAMPLES

Final Products

### 1. [1-Benzyl(4-piperidyl)]{2-[(2-pyridylmethyl)amino]-5-(3-thienyl)pyrimidin-4-yl}amine

3-Thiopheneboronic acid (0.62 g, 4.85 mmol) was added to a solution of {5-bromo-2-[(2-pyridylmethyl)-amino]pyrimidin-4-yl}[1-benzyl(4-piperidyl)]amine (1.00 g, 2.21 mmol) in ethylene glycol dimethyl ether (50 mL). A solution of potassium carbonate (1.25 g, 9.04 mmol) in water (15 mL) was added to the above reaction mixture. Tetrakis(triphenylphosphine)palladium (260 mg, 0.225 mmol) was added to the reaction and stirred at 80° C. under nitrogen for 2 hours. The reaction mixture was diluted with water (150 mL) and extracted with methylene chloride (4×100 mL). The organic layer was concentrated and the residue was purified using flash chromatography (5% methanol in ethyl acetate) to give [1-benzyl(4-piperidyl)]{2-[(2-pyridylmethyl)amino]-5-(3-thienyl)pyrimidin-4-yl}amine (0.45 g, 45% yield) as an off-white foam; ESMS 457 (M+1)⁺.

### 2. {5-(4-Methoxyphenyl)-2-[(4-pyridylmethyl)amino)pyrimidin-4-yl}[1-benzyl(4-piperidyl)]amine

The title compound was prepared from {5-bromo-2-[(4-pyridylmethyl)amino)pyrimidin-4-yl}[1-benzyl(4-piperidyl)]amine, 4-methoxyphenyl boronic acid as described in Example 1 as an off-white foam; ESMS 481 (M+1)⁺.

### 3. {5-Phenyl-2-[(4-pyridylmethyl)amino)pyrimidin-4-yl}[1-benzyl(4-piperidyl)]amine

The title compound was prepared from {5-bromo-2-[(4-pyridylmethyl)amino)pyrimidin-4-yl}[1-benzyl(4-piperidyl)]amine, phenyl boronic acid as described in Example 1 as an off-white foam; ESMS 451 (M+1)⁺.

### 4. {5-(4-Chlorophenyl)-2-[(4-pyridylmethyl)amino)pyrimidin-4-yl}[1-benzyl(4-piperidyl)]amine

The title compound was prepared from {5-bromo-2-[(4-pyridylmethyl)amino)pyrimidin-4-yl}[1-benzyl(4-piperidyl)]amine, 4-chlorophenyl boronic acid as described in Example 1 as an off-white foam; ESMS 486 (M+1)⁺.

### 5. {5-(4-(N,N-Dimethylamino)phenyl)-2-[(4-pyridylmethyl)amino)pyrimidin-4-yl}[1-benzyl(4-piperidyl)]amine

The title compound was prepared from {5-bromo-2-[(4-pyridylmethyl)amino)pyrimidin-4-yl}[1-benzyl(4-piperidyl)]amine, 4-(N,N-dimethylamino)phenyl boronic acid as described in Example 1 as an off-white foam; ESMS 494 (M+1)⁺.

### 6. {5-(Phenyl-4-carboxamido)-2-[(4-pyridylmethyl)amino)pyrimidin-4-yl}[1-benzyl(4-piperidyl)]-amine

The title compound was prepared from {5-bromo-2-[(4-pyridylmethyl)amino)pyrimidin-4-yl}[1-benzyl(4-piperidyl)]amine, 4-carboxyphenylboronic acid as described in Example 3 to provide {5-(4-carboxyphenyl)-2-[(4-pyridylmethyl)amino)pyrimidin-4-yl}[1-benzyl(4-piperidyl)]amine. A solution of {5-(4-carboxy-phenyl)-2-[(4-pyridylmethyl)amino)pyrimidin-4-yl}[1-benzyl(4-piperidyl)]amine, carbonyl diimidazole (1.2 eq), ammonium hydroxide (12 eq) in tetrahydrofuran (25 mL) was stirred at room temperature for 6 h. Flash chromatography (SiO₂, 5% methanol in ethyl acetate) afforded the title compound as white solid; ESMS 494 (M+1)⁺.

### 7. {5-(4-Carboxyphenyl)-2-[(4-pyridylmethyl)amino)pyrimidin-4-yl}[1-benzyl(4-piperidyl)]amine

The title compound was prepared from {5-bromo-2-[(4-pyridylmethyl)amino)pyrimidin-4-yl}[1-benzyl(4-piperidyl)]amine, 4-carboxyphenylboronic acid as described in Example 1 to provide the title compound; ESMS 495 (M+1)⁺.

### 8. {5-(2-Thienyl)-2-[(4-pyridylmethyl)amino)pyrimidin-4-yl}[1-benzyl(4-piperidyl)]amine

The title compound was prepared from (5-bromo-2-[(4-pyridylmethyl)amino)pyrimidin-4-yl)[1-benzyl(4-piperidyl)]amine, 2-thienylboronic acid as described in Example 1 to provide the title compound; ESMS 457 (M+1)⁺.

### 9. {5-(2-Furanyl)-2-[(4-pyridylmethyl)amino)pyrimidin-4-yl}[1-benzyl(4-piperidyl)]amine

The title compound was prepared from {5-bromo-2-[(4-pyridylmethyl)amino)pyrimidin-4-yl}[1-benzyl(4-piperidyl)]amine, 2-furanylboronic acid as described in Example 1 to provide the title compound; ESMS 441 (M+1)⁺.

### 10. {5-(3-Furanyl)-2-[(4-pyridylmethyl)amino)pyrimidin-4-yl}[1-benzyl(4-piperidyl)]amine

The title compound was prepared from {5-bromo-2-[(4-pyridylmethyl)amino)pyrimidin-4-yl}[1-benzyl(4-piperidyl)]amine, 3-furanylboronic acid as described in Example 1 to provide the title compound; ESMS 441 (M+1)⁺.

### 11. {5-(3-Thienyl)-2-[(4-pyridylmethyl)amino)pyrimidin-yl}[1-benzyl(4-piperidyl)]amine

The title compound was prepared from {5-bromo-2-(4-pyridylmethyl)amino)pyrimidin-4-yl}[1-benzyl(4-piperidyl)]amine, 3-thienylboronic acid as described in Example 1 to provide the title compound; ESMS 457 (M+1)⁺.

### 12. {5-(2-Furanyl)-2-[(2-pyridylmethyl)amino)pyrimidin-4-yl}[1-benzyl(4-piperidyl)]amine

The title compound was prepared from {5-bromo-2-[(2-pyridylmethyl)amino)pyrimidin-4-yl}[1-benzyl(4-piperidyl)]amine, 2-furanylboronic acid as described in Example 1 to provide the title compound; ESMS 441 (M+1)⁺.

### 13. {5-(3-Furanyl)-2-[(2-pyridylmethyl)amino)pyrimidin-4-yl}[1-benzyl(4-piperidyl)]amine

The title compound was prepared from {5-bromo-2-[(2-pyridylmethyl)amino)pyrimidin-4-yl}[1-benzyl(4-piperidyl)]amine, 3-furanylboronic acid as described in Example 1 to provide the title compound; ESMS 441 (M+1)⁺.

### 14. {5-(2-Thienyl)-2-[(2-pyridylmethyl)amino)pyrimidin-4-yl}[1-benzyl(4-piperidyl)]amine

The title compound was prepared from {5-bromo-2-[(2-pyridylmethyl)amino)pyrimidin-4-yl}[1-benzyl(4-piperidyl)]amine, 2-thienylboronic acid as described in Example 1 to provide the title compound; ESMS 457 (M+1)⁺.

### 15. {5-(Phenyl-4-carboxamido)-2-[2-pyridylmethyl)amino)pyrimidin-4-yl}[1-benzyl(4-piperidyl)]amine

The title compound was prepared from {5-bromo-2-[(2-pyridylmethyl)amino)pyrimidin-4-yl}[1-benzyl(4-piperidyl)]amine, 4-carboxyphenylboronic acid as described in Example 3 to provide {5-(phenyl-4-carboxy)-2-[(2-pyridylmethyl)amino)pyrimidin-4-yl)[1-benzyl(4-piperidyl)]amine. A solution of {5-(phenyl-4-carboxy)-2-[(4-pyridylmethyl)amino)pyrimidin-4-yl)[1-benzyl(4-piperidyl)]amine, carbonyl diimidazole (1.2 eq), ammonium hydroxide (12 eq) in tetrahydrofuran (0.1M) was stirred at room temperature for 6 h. Flash chromatography (SiO2, 5% methanol in ethyl acetate) afforded the title compound as white solid; ESMS 494 (M+1)⁺.

### 16. N(4)-(1-Benzyl-piperidin-4-yl)-5-(3-chloro-4-fluoro-phenyl)-N(2)-pyridin-2-ylmethyl-pyrimidine-2,4-diamine

The title compound was prepared from (5-bromo-2-[(4-pyridylmethyl)amino)pyrimidin-4-yl}[1-benzyl(4-piperidyl)]amine, 3-chloro-4-fluoro-phenyl boronic acid as described in Example 1 as an off-white foam; ESMS 503 (M+1)⁺.

### 17. N-(3-[4-(1-Benzyl-piperidin-4-ylamino)-2-[(pyridin-2-ylmethyl)-amino]-pyrimidin-5-yl]phenyl)-acetamide

The title compound was prepared from (5-bromo-2-[(4-pyridylmethyl)amino)pyrimidin-4-yl)[1-benzyl(4-piperidyl)]amine, 3-acetaminophenyl boronic acid as described in Example 1 as an off-white foam; ESMS 508 (M+1)⁺.

### 18. 5-Benzo[1,3]dioxol-5-yl-N(4)-(1-benzyl-piperidin-4-yl)-N(2)-pyridin-2-ylmethyl-pyrimidine-2,4-diamine

The title compound was prepared from {5-bromo-2-[(4-pyridylmethyl)amino)pyrimidin-4-yl}[1-benzyl(4-piperidyl)]amine, 5-benzo[1,3]dioxolo boronic acid as described in Example 1 as an off-white foam; ESMS 495 (M+1)⁺.

### 19. 3-[4-(1-Benzyl-piperidin-4-ylamino)-2-[(pyridin-2-ylmethyl)-amino]-pyrimidin-5-yl]-phenol

The title compound was prepared from (5-bromo-2-[(4-pyridylmethyl)amino)pyrimidin-4-yl}[1-benzyl(4-piperidyl)]amine, 3-hydroxyphenylphenyl boronic acid as described in Example 1 as an off-white foam; ESMS 467 (M+1)⁺.

### 20. 4-{4-(1-Benzyl-piperidin-4-ylamino)-2-[(pyridin-2-ylmethyl)-amino]-pyrimidin-5-yl}-phenol

The title compound was prepared from {5-bromo-2-[(4-pyridylmethyl)amino)pyrimidin-4-yl}[1-benzyl(4-piperidyl)]amine, 4-hydroxyphenyl boronic acid as described in Example 1 as an off-white foam; ESMS 467 (M+1)⁺.

### 21. 1-(4{(4-(1-Benzyl-piperidin ylamino)-2-[(pyridin-2-ylmethyl)-amino]-pyrimidin-5-yl}phenyl)-ethanone

The title compound was prepared from {5-bromo-2-[(4-pyridylmethyl)amino)pyrimidin-4-yl)[1-benzyl(4-piperidyl)]amine, 4-acetylphenyl boronic acid as described in Example 1 as an off-white foam; ESMS 493 (M+1)⁺.

### 22. 1-(3-[4-(1-Benzyl-piperidin ylamino)-2-[(pyridin-2-ylmethyl)-amino]-pyrimidin-5-yl}phenyl)-ethanone

The title compound was prepared from {5-bromo-2-[(4-pyridylmethyl)amino)pyrimidin-4-yl}[1-benzyl(4-piperidyl)]amine, 3-acetylphenyl boronic acid as described in Example 1 as an off-white foam; ESMS 493 (M+1)⁺.

### 23. 4-{4-(1-Benzyl piperidin-4-ylamino)-2-[(pyridin-2-ylmethyl)-amino]-pyrimidin-5-yl}N,N-di-methyl-benzamide

The title compound was prepared from {5-bromo-2-[(4-pyridylmethyl)amino)pyrimidin-4-yl}[1-benzyl(4-piperidyl)]amine, 4-dimethylaminocarbonylphenyl boronic acid as described in Example 1 as an off-white foam; ESMS 522 (M+1)⁺.

### 24. N-(4-[4-(1-Benzyl-piperidin-4-ylamino)-2-[(pyridin-2-ylmethyl)-amino]-pyrimidin-5-yl]-phenyl)-acetamide

The title compound was prepared from {5-bromo-2-[(4-pyridylmethyl)amino)pyrimidin-4-yl}[1-benzyl(4-piperidyl)]amine, 4-acetaminophenyl boronic acid as described in Example 1 as an off-white foam; ESMS 508 (M+1)⁺.

Intermediates

### A. (5-Bromo-2-chloropyrimidin-4-yl)[1-benzyl (4-piperidyl)]amine

4-Amino-1-benzylpiperidine (4.09 g, 0.22 mol) was added to a solution of 5-bromo-2, 4-dichloropyrimidine (4.90 g, 0.22 mol) and potassium carbonate (3.86 g, 0.28 mol) in THF (150 mL) under constant stirring at room temperature. The reaction was stirred for 30 min at room temperature then diluted with water (400 mL) and extracted with ethyl acetate (2×200 mL). The organic layer was separated and evaporated under reduced pressure to give a residue. The residue was purified using flash chromatography (SiO₂, ethyl acetate) to give (5-bromo-2-chloropyrimidin-4-yl)[1-benzyl(4-piperidyl)]amine (5.15 g, 65% yield) as clear oil; ESMS 381 (M+1)⁺.

### B. Ethyl 4-({5-bromo-2-chloropyrimidin-4-yl}amino)piperidinecarboxylate

The title compound was prepared from ethyl 4-aminopiperdinecarboxylate and 5-bromo-2,4-dichloro-pyrimidine as described in Example A to give the title compound; ESMS 363 (M+1)⁺.

### C. {5-Bromo-2-[(2-pyridylmethyl)amino]pyrimidin-4-yl}[1-benzyl(4-piperidyl)]amine

A solution of (5-bromo-2-chloropyrimidin-4-yl)[1-benzyl(4-piperidyl)]amine (4.91 g, 0.13 mol) and 2-(aminomethyl)pyridine (3.20 g, 0.30 mol) was heated (neat) at 120° C. for 25 minutes. The reaction mixture was partitioned between ethyl acetate (300 mL) and saturated aqueous NaHCO₃ solution (300 mL). The organic layer was separated, washed with brine, concentrated, and purified using flash chromatography (10% methanol in ethyl acetate) to give {5-bromo-2-[(2-pyridylmethyl)amino]pyrimidin-4-yl}[1-benzyl(4-piperidyl)]amine (3.18 g 55% yield) as off-white foam; ESMS 453 (M+1)⁺.

### D. {5-Bromo-2-[(4-pyridylmethyl)amino]pyrimidin-4-yl}[1-benzyl(4-piperidyl)]amine

The title compound was prepared from (5-bromo-2-chloropyrimidin-4-yl)[1-benzyl(4-piperidyl)]amine and 4-(aminomethyl)pyridine as described in Example C to provide an off-white foam; ESMS 453 (M+1)⁺.

### E. Ethyl 4-({5-bromo-2-[(2-pyridylmethyl)amino]pyrimidin-4-yl}amino)piperdinecarboxylate

The title compound was prepared from Ethyl 4-({5-bromo-2-chloropyrimidin-4-yl}amino)piperidine-carboxylate and 2-(aminomethyl)pyridine as described in Example C to give the title compound; ESMS 453 (M+1)⁺.

Biological Investigations

Protein kinase C-theta is a member of the Ca²⁺-independent novel protein kinase C (PKC) subfamily, which is predominantly expressed in skeletal muscle and T-cells (Baler et al., JBC 268:4997; Bauer et al., Eur J. Immunol 30: 3645). PKC-θ was shown to selectively colocalize with the TCR to the T cell synapse when antigen-specific T cells are engaged by their physiological ligand (Monks et al., Nature 395:82; Monks et al., Nature 385:83). Functional studies of PKC-θ revealed an early and essential role in the TCR/CD28-induced stimulation of MAP kinase JNK/AP-1 and NFAT, but also the IKKβ/I-κB/NF-κB signaling cascade (see for review Altman et al., Immunol Today 21:567; Bauer and Baier, 2002 Mol. Immunol., submitted).

In T cells PKC-θ activates AP-1, NFAT and NF-κB (Bauer et al., Eur. J. Immunol. 30: 3645; Lin et al., Mol Cell Biol 20:2933; Coudronniere et al., PNAS 97:3394) and PKC-θ was shown to synergize with Calcineurin in inducing the IL-2 gene (Werlen et al., EMBO J. 17:3101; Ghaffari-Tabrizi et al., Eur. J. Immunol. 29:132). Inhibition of PKC-θ leads to impaired T-cell functions (Baier-Bitterlich et al., Mol Cell Biol 16:1842; Ghaffari-Tabrizi et al., Eur. J. Immunol. 29:132). Consistently, T-cells of PKC-θ-deficient mice display profound defects in TCR-induced IL-2 production and, subsequently, T-cell proliferation (Sun et al, Nature 404:402; Pfeifhofer et al., submitted).

For the investigation of PKC-θ inhibition on the enzymatic level the phosphorylation of a substrate pep-tide by recombinant PKC-θ enzyme can be measured. On the cellular level (in vitro) the immunomodulatory potential of PKC-θ inhibitors is evident from the inhibition of activated T-cell responses such as proliferation, cytokine synthesis (e.g. IL2) and expression of activation markers. Substances, which inhibit the aforementioned proinflammatory parameters are those which inhibit PKC-E.

Protein Kinase C-θ Assay

The compounds of formula 1 were tested for their activity on PKC-θ according to the following method. The assay was performed in 96 well microtiter plates (Perkin Elmer Wallac) at a final assay volume of 200 μl. The reaction mixture (50 μl) contained 10 μl of recombinant human PKC-θ enzyme together with 5 μl of the test compound and 3 μM biotinylated PKC-θ substrate peptide (Biotin-RKRQRSMRRRVHOH), 160 μM phosphatidylserine, 0.3 mg/ml BSA, 1 μM ATP and 2 μCi of ³³γ-ATP (Amersham) in 40 mM Tris-buffer pH 7.4. Incubation was performed for 40 min at room temperature. The reaction was stopped by adding 150 μl of a mixture containing 10 mM ATP and 1.33 mg/ml streptavidin coated yttrium silicate SPA beads (Amersham). Incorporated radioactivity (cpm) was measured for 2 min in a radioactivity counter (Wallac MicroBeta JET). According to the method described above IC₅₀ measurement was performed on a routine basis by incubating a serial dilution of inhibitor at the desired concentrations and a final DMSO concentration of 1% (v/v), which did not affect PKC-θ activity. IC₅₀ values for inhibition of PKC-θ were calculated from the concentration-inhibition curves by nonlinear-regression.

The inhibitory values determined for the compounds according to the invention follow from the following table A, in which the numbers of the compounds correspond to the numbers of the examples.

TABLE A
Inhibition of PKC-θ activity [measured as IC₅₀ (μmol/l)]

| Example No. | PKC-θ |
|---|---|
| 1 | <4.0 |
| 12 | <4.0 |
| 13 | <4.0 |
| 14 | <4.0 |
| 15 | <4.0 |
| 16 | <4.0 |
| 17 | <4.0 |
| 18 | <4.0 |
| 19 | <4.0 |
| 20 | <4.0 |
| 21 | <4.0 |
| 22 | <4.0 |
| 23 | <4.0 |
| 24 | <4.0 |

To determine the effects of compounds of formula 1 on T-cell activation the following assays were performed.

CD4+ Proliferation Assay

CD4+ lymphocytes were purified as described by Hatzelmann and Schudt (J Pharmacol Exp Ther 297: 267-279) and resuspended in assay medium (RPMI 1640/10% fetal calf serum (FCS) containing 2 mM Glutamine, 1% sodiumpyruvate, 1% non-essential amino acids and 1% penicillin/streptomycin) at a density of 1×10⁶ cells/ml. 96 well plates were coated with αCD3 antibodies (0.3 μg/well; Ortho-clone OKT-3, Jansen-Cilag) for 2.5 h at 37° C. in 5% CO₂ and then washed twice with PBS (200 μl/well). Prior to plating of the cells (200 μl, 2×10⁵ cells) the compounds of formula 1 dissolved in 2% DMSO were added to the antibody-coated plates at the desired concentrations. Following a preincubation period of 30 min at 37° C. and 5% CO₂ 10 μl of αCD28 antibody (3 μg/ml, Beckman) were added and incubation continued for 48 h at 37° C. and 5% CO₂. 18 h prior to cell harvest 10 μl of ³H-methylthymidin (0.2 μCi, Amersham) were added. After 48 h total incubation time the cells were lysed using deionized water and radiolabeled DNA was immobilized on 96 well filter plates using a Tomtec device. The plates were dried at 60° C. for 1 h and overlayed with 40 μl Microscint-O (Packard) before counting in a Topcount radioactivity counter (Packard). Calculation of IC50 values was performed as described above.

The inhibitory values determined for the compounds according to the invention follow from the following table B, in which the numbers of the compounds correspond to the numbers of the examples.

TABLE B
Inhibition of CD4+ cell proliferation [measured as IC₅₀ (μmol/l)]

| | CD4+ |
|---|---|
| Example No. | proliferation |
| 1 | <4.0 |
| 8 | <4.0 |
| 14 | <4.0 |
| 15 | <4.0 |

CD4+ IL-2 Secretion Assay

CD4+ T lymphocytes were stimulated and treated with compounds of formula 1 as described above for the CD4+ proliferation assay. Following a 48 h incubation period IL-2 levels in the supernatants (50 μl/well) were determined by ELISA (Beckman Coulter). Calculation of IC₅₀ values was performed as described above.

The inhibitory values on CD4+ T-cell activation determined for the compounds according to the invention follow from the following table C, in which the numbers of the compounds correspond to the numbers of the examples.

TABLE C
Inhibition of IL-2 secretion in CD4+ cell [measured as IC₅₀ (μmol/l)]

| | CD4+ IL2 |
|---|---|
| Example No. | proliferation |
| 1 | <4.0 |
| 6 | <4.0 |
| 8 | <4.0 |
| 12 | <4.0 |
| 13 | <4.0 |
| 14 | <4.0 |
| 15 | <4.0 |

The invention being thus described, it will be obvious that the same may be varied in many ways. Such variations are not to be regarded as a departure from the spirit and scope of the invention and all such modifications are intended to be included within the scope of the following claims.

1. A compound of formula 1, 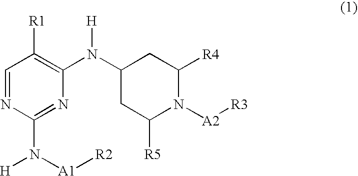
in which
R1 is a mono- or bicyclic aromatic radical substituted by R11, R12, R13 and R14, wherein R1 is selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl(thienyl), benzothiophenyl (benzothienyl), thiazolyl, isoxazolyl, pyridinyl, pyrimidinyl, quinolinyl and isoquinolinyl, where
R11 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R12 methylenedioxy or ethylenedioxy,
R12 is hydrogen, 1-4C-alkyl, 1-4C-alkoxy, 1-4C-alkoxycarbonyl, halogen, trifluoromethyl or hydroxyl, or together with R11 methylenedioxy or ethylenedioxy,
R13 is hydrogen, 1-4C-alkyl or halogen and
R14 is hydrogen, 1-4C-alkyl or halogen,
where
aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R2 is a mono- or bicyclic aromatic radical substituted by R21, R22, R23 and R24, wherein R2 is selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl(thienyl), benzothiophenyl (benzothienyl), thiazolyl, isoxazolyl, pyridinyl, pyrimidinyl, quinolinyl and isoquinolinyl, where
R21 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R22 methylenedioxy or ethylenedioxy,
R22 is hydrogen, 1-4C-alkyl, 1-4C-alkoxy, 1-4C-alkoxycarbonyl, halogen, trifluoromethyl or hydroxyl, or together with R21 methylenedioxy or ethylenedioxy,
R23 is hydrogen, 1-4C-alkyl or halogen and
R24 is hydrogen, 1-4C-alkyl or halogen,
where
aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R3 is a mono- or bicyclic aromatic radical substituted by R31, R32, R33 and R34, wherein R3 is selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl(thienyl), benzothiophenyl (benzothienyl), thiazolyl, isoxazolyl, pyridinyl, pyrimidinyl, quinolinyl and isoquinolinyl, where
R31 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R32 methylenedioxy or ethylenedioxy,
R32 is hydrogen, 1-4C-alkyl, 1-4C-alkoxy, 1-4C-alkoxycarbonyl, halogen, trifluoromethyl or hydroxyl, or together with R31 methylenedioxy or ethylenedioxy,
R33 is hydrogen, 1-4C-alkyl or halogen and
R34 is hydrogen, 1-4C-alkyl or halogen,
where
aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R4 is hydrogen or methyl,
R5 is hydrogen or methyl,
A1 is 1-3C-alkylene or ethyleneoxy (—CH₂—CH₂—O—) and
A2 is 1-3C-alkylene or ethyleneoxy (—CH₂—CH₂—O—), or a hydrate, solvate, salt, hydrate of a salt or solvate of a salt thereof.

2. A compound of formula 1 according to claim 1,
in which
R1 is a mono- or bicyclic aromatic radical substituted by R11, R12, R13 and R14, wherein R1 is selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl(thienyl), benzothiophenyl (benzothienyl), thiazolyl, isoxazolyl, pyridinyl, pyrimidinyl, quinolinyl and isoquinolinyl, where
R11 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R12 is hydrogen, 1-4C-alkyl, 1-4C-alkoxy, 1-4C-alkoxycarbonyl, halogen, trifluoromethyl or hydroxyl,
R13 is hydrogen, 1-4C-alkyl or halogen and
R14 is hydrogen, 1-4C-alkyl or halogen,
where
aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R2 is a mono- or bicyclic aromatic radical substituted by R21, R22, R23 and R24, wherein R2 is selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl(thienyl), benzothiophenyl (benzothienyl), thiazolyl, isoxazolyl, pyridinyl, pyrimidinyl, quinolinyl and isoquinolinyl,
where
R21 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R22 is hydrogen, 1-4C-alkyl, 1-4C-alkoxy, 1-4C-alkoxycarbonyl, halogen, trifluoromethyl or hydroxyl,
R23 is hydrogen, 1-4C-alkyl or halogen and
R24 is hydrogen, 1-4C-alkyl or halogen,
where
aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R3 is a mono- or bicyclic aromatic radical substituted by R31, R32, R33 and R34, wherein R3 is selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl(thienyl), benzothiophenyl (benzothienyl), thiazolyl, isoxazolyl, pyridinyl, pyrimidinyl, quinolinyl and isoquinolinyl, where
R31 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R32 is hydrogen, 1-4C-alkyl, 1-4C-alkoxy, 1-4C-alkoxycarbonyl, halogen, trifluoromethyl or hydroxyl,
R33 is hydrogen, 1-4C-alkyl or halogen and
R34 is hydrogen, 1-4C-alkyl or halogen,
where
aryl is phenyl or substituted phenyl having one, two or three substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl, cyano and mixtures thereof,
R4 is hydrogen,
R5 is hydrogen,
A1 denotes 1-3C-alkylene and
A2 denotes 1-3C-alkylene,
or a hydrate, solvate, salt, hydrate of a salt or solvate of a salt thereof.

3. A compound of formula 1 according to claim 1,
in which
R1 is an aromatic radical substituted by R11, R12, R13 and R14, wherein R1 is selected from the group consisting of phenyl, furanyl (furyl) and thiophenyl (thienyl), where
R11 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl, or together with R12 methylenedioxy or ethylenedioxy,
R12 is hydrogen or halogen, or together with R11 methylenedioxy or ethylenedioxy,
R13 is hydrogen and
R14 is hydrogen,
R2 is an aromatic radical substituted by R21, R22, R23 and R24, wherein R2 is selected from the group consisting of pyridinyl and pyrimidinyl,
where
R21 is hydrogen, 1-4C-alkyl, 1-4C-alkoxy or halogen,
R22 is hydrogen or halogen,
R23 is hydrogen and
R24 is hydrogen,
R3 is an aromatic radical substituted by R31, R32, R33 and R34, wherein R3 is selected from the group consisting of phenyl and pyridinyl,
where
R31 is hydrogen, 1-4C-alkyl, hydroxy-1-4C-alkyl, 1-4C-alkoxy, 2-4C-alkenyloxy, 1-4C-alkylcarbonyl, carboxyl, aminocarbonyl, mono- or di-1-4C-alkylaminocarbonyl, 1-4C-alkoxycarbonyl, carboxy-1-4C-alkyl, 1-4C-alkoxycarbonyl-1-4C-alkyl, halogen, hydroxyl, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R32 is hydrogen or halogen,
R33 is hydrogen and
R34 is hydrogen,
R4 is hydrogen or methyl,
R5 is hydrogen or methyl,
A1 is 1-3C-alkylene or ethyleneoxy (—CH₂—CH₂—O—) and
A2 is 1-3C-alkylene or ethyleneoxy (—CH₂—CH₂—O—),
or a hydrate, solvate, salt, hydrate of a salt or solvate of a salt thereof.

4. A compound of formula 1 according to claim 1, in which
R1 is an aromatic radical substituted by R11, R12, R13 and R14, wherein R1 is selected from the group consisting of phenyl, furanyl (furyl) and thiophenyl (thienyl),
where
R11 is hydrogen, 1-4C-alkoxy, 1-4C-alkylcarbonyl, carboxyl, aminocarbonyl, mono- or di-1-4C-alkylaminocarbonyl, halogen, hydroxyl or mono- or di-1-4C-alkylamino, or together with R12 methylenedioxy or ethylenedioxy,
R12 is hydrogen or halogen, or together with R11 methylenedioxy or ethylenedioxy,
R13 is hydrogen and
R14 is hydrogen,
R2 is an aromatic radical substituted by R21, R22, R23 and R24, wherein R2 is selected from the group consisting of pyridinyl and pyrimidinyl, where
R21 is hydrogen,
R22 is hydrogen,
R23 is hydrogen and
R24 is hydrogen,
R3 is an aromatic radical substituted by R31, R32, R33 and R34, wherein R3 is selected from the group consisting of phenyl and pyridinyl, where
R31 is hydrogen, 1-4C-alkoxy or halogen,
R32 is hydrogen,
R33 is hydrogen and
R34 is hydrogen,
R4 is hydrogen or methyl,
R5 is hydrogen or methyl,
A1 denotes methylene, ethylene, ethylidene [—CH(CH₃)—] or ethyleneoxy (—CH₂—CH₂—O—) and
A2 denotes methylene, ethylene, ethylidene [—CH(CH₃)—] or ethyleneoxy (—CH₂—CH₂—O—),
or a hydrate, solvate, salt, hydrate of a salt or solvate of a salt thereof.

5. A compound of formula 1 according to claim 1,
in which
R1 is an aromatic radical substituted by R11, R12, R13 and R14, wherein R1 is selected from the group consisting of phenyl, furanyl (furyl) and thiophenyl (thienyl),
where
R11 is hydrogen, 1-4C-alkoxy, 1-4C-alkylcarbonyl, carboxyl, aminocarbonyl, mono- or di-1-4C-alkylaminocarbonyl, halogen, hydroxyl or mono- or di-1-4C-alkylamino, 1-4C-alkylcarbonylamino, or together with R12 methylenedioxy or ethylenedioxy,
R12 is hydrogen or halogen, or together with R11 methylenedioxy or ethylenedioxy,
R13 is hydrogen and
R14 is hydrogen,
R2 is an aromatic radical substituted by R21, R22, R23 and R24, wherein R2 is selected from the group consisting of pyridinyl and pyrimidinyl, where
R21 is hydrogen,
R22 is hydrogen,
R23 is hydrogen and
R24 is hydrogen,
R3 is an aromatic radical substituted by R31, R32, R33 and R34, wherein R3 is selected from the group consisting of phenyl and pyridinyl, where
R31 is hydrogen, 1-4C-alkoxy or halogen,
R32 is hydrogen,
R33 is hydrogen and
R34 is hydrogen,
R4 is hydrogen or methyl,
R5 is hydrogen or methyl,
A1 denotes methylene, ethylene, ethylidene (—CH(CH₃)—] or ethyleneoxy (—CH₂—CH₂—O—) and
A2 denotes methylene, ethylene, ethylidene [—CH(CH₃)—] or ethyleneoxy (—CH₂—CH₂—O—),
or a hydrate, solvate, salt, hydrate of a salt or solvate of a salt thereof.

6. A compound of formula 1 according to claim 1, in which
R1 is furanyl (furyl), thiophenyl(thienyl) or phenyl substituted by R11 and R12, where
R11 is hydrogen, 1-4C-alkoxy, 1-4C-alkylcarbonyl, carboxyl, aminocarbonyl, mono- or di-1-4C-alkylaminocarbonyl, halogen, hydroxyl or mono- or di-1-4C-alkylamino, 1-4C-alkylcarbonylamino, or together with R12 methylenedioxy or ethylenedioxy,
R12 is hydrogen or halogen, or together with R11 methylenedioxy or ethylenedioxy,
R2 is pyridinyl,
R3 is phenyl,
R4 is hydrogen,
R5 is hydrogen,
A1 denotes methylene and
A2 denotes methylene,
or a hydrate, solvate, salt, hydrate of a salt or solvate of a salt thereof.

7. A compound of formula 1 according to claim 1, in which
R1 is furanyl (furyl), thiophenyl(thienyl) or phenyl substituted by R11 and R12,
where
R11 is hydrogen, 1-4C-alkoxy, carboxyl, aminocarbonyl, halogen or di-1-4C-alkylamino and R12 is hydrogen,
R2 is pyridinyl,
R3 is phenyl,
R4 is hydrogen,
R5 is hydrogen,
A1 denotes methylene and
A2 denotes methylene,
or a hydrate, solvate, salt, hydrate of a salt or solvate of a salt thereof.

8. A compound of formula 1 according to claim 1, wherein R2 is 2-pyridinyl or 4-pyridinyl, or a hydrate, solvate, salt, hydrate of a salt or solvate of a salt thereof.

9. A compound of formula 1 according to claim 1, wherein R3 is phenyl, or a hydrate, solvate, salt, hydrate of a salt or solvate of a salt thereof.

10. A compound of formula 1 according to claim 1, wherein R1 is furanyl (furyl), thiophenyl (thienyl) or phenyl substituted by R11 and R12, where R11 is hydrogen, 1-4C-alkoxy, carboxyl, aminocarbonyl, halogen or di-1-4C-alkylamino and R12 is hydrogen, or a hydrate, solvate, salt, hydrate of a salt or solvate of a salt thereof.

11. A compound of formula 1 according to claim 1, wherein R1 is furanyl (furyl), thiophenyl (thienyl) or phenyl substituted by R11 and R12, where R11 is hydrogen, 1-4C-alkoxy, halogen, di-1-4C-alkylamino, aminocarbonyl, carboxyl, 1-4C-alkylcarbonylamino, hydroxyl, 1-4C-alkylcarbonyl or together with R12 methylenedioxy or ethylenedioxy and R12 is hydrogen, halogen or together with R11 methylenedioxy or ethylenedioxy, or a hydrate, solvate, salt, hydrate of a salt or solvate of a salt thereof.

12. A compound of formula 1 according to claim 1, wherein R1 is 2-furanyl or 3-furanyl, or a hydrate, solvate, salt, hydrate of a salt or solvate of a salt thereof.

13. A compound of formula 1 according to claim 1, wherein R1 is 2-thiophenyl or 3-thiophenyl, or a hydrate, solvate, salt, hydrate of a salt or solvate of a salt thereof.

14. A compound of formula 1 according to claim 1, wherein R1 is selected from the group consisting of phenyl, 4-methoxyphenyl, 4-chlorophenyl, 4-dimethylaminophenyl, 4-aminocarbonylphenyl, 4-carboxyphenyl, 3-chloro-4-fluorophenyl, 3-acetylaminophenyl, benzo[1,3]dioxol-5-yl, 3-hydroxyphenyl, 4-hydroxyphenyl, 4-acetylphenyl, 3-acetylphenyl, 4-acetylaminophenyl, 4-dimethylaminocarbonyl-phenyl and 4-aminocarbonylphenyl, or a hydrate, solvate, salt, hydrate of a salt or solvate of a salt thereof.

15. A compound of formula 1 according to claim 1, wherein A1 denotes methylene and A2 denotes methylene, or a hydrate, solvate, salt, hydrate of a salt or solvate of a salt thereof.

16. A pharmaceutical composition comprising a compound of the formula 1 as claimed in claim 1 and/or a pharmaceutically acceptable hydrate, solvate, salt, hydrate of a salt or solvate of a salt thereof together with a pharmaceutically acceptable excipient and/or carrier.

17. A method of treating a patient afflicted with a disease or disorder, comprising the step of administering a therapeutically effective amount of a compound of the formula 1 as claimed in claim 1 and/or a pharmaceutically acceptable hydrate, solvate, salt, hydrate of a salt or solvate of a salt thereof to said patient afflicted with said disease or disorder, wherein the disease or disorder is selected from the group consisting of an acute and/or chronic airway disorder, an inflammatory or allergen-induced airway disorder, bronchitis, obstructive bronchitis, spastic bronchitis, allergic bronchitis, allergic asthma, bronchial asthma, emphysema, chronic obstructive pulmonary disease (COPD), a disorder which is based on an excessive release of T-Cell derived cytokines, HIV-infection, septic shock, adult respiratory distress syndrome, graft-versus-host reactions, acute or chronic rejection of organ or tissue allo- or xenografts, generalized inflammations in the gastrointestinal area, rheumatoid arthritis, rheumatoid spondylitis, osteoarthritis, faulty immunological reactions in the area of the upper airways and the adjacent regions, dermatoses of the proliferative, inflammatory or allergic type, psoriasis (vulgaris), toxic and allergic contact eczema, atopic eczema, seborrheic eczema, lichen simplex, sunburn, pruritus in the anogenital area, alopecia areata, hypertrophic scars, discoid lupus erythematosus, follicular and wide-area pyodermias, endogenous and exogenous acne, acne rosacea, other proliferative, inflammatory, allergic skin disorders, a disorder in connection with disturbances of brain metabolism, disorders of the central nervous system (CNS), cerebral senility, senile dementia, multiinfarct dementia, depression, arteriosclerotic dementia, cancer and diabetes insipidus.

18. A method according to claim 17, in which a compound of claim 1 and/or a hydrate, solvate, salt, hydrate of a salt or solvate of a salt thereof, is used in combination with other therapeutic agents used in clinical practice for the treatment of the said disease or disorder.

19. A process for preparing a compound of formula 1 as claimed in claim 1 or a hydrate, solvate, salt, hydrate of a salt or solvate of a salt thereof, which comprises reacting a boronic acid derivative R1-B(OH)₂ wherein R1 has the meaning specified in claim 1, with a pyrimidine derivate of formula (4) 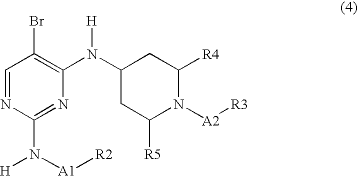
in which A1, A2, R2, R3, R4 and R5 have a meaning specified in claim 1, and optionally converting an obtained compound into a corresponding hydrate, solvate, salt, hydrate of a salt or solvate of a salt, or converting an obtained hydrate, solvate, salt, hydrate of a salt or solvate of a salt into a corresponding free compound.
